(19) United States
(12) Patent Application Publication
(10) Pub. No.: US 2005/0267493 A1
(43) Pub. Date: Dec. 1, 2005

(54) Method and system for tissue repair using dual catheters

(76) Inventors: Stefan G. Schreck, Vista, CA (US); William J. Allen, Stratford, CT (US); Scott Reed, Monroe, CT (US); Alan B. Bachman, New Haven, CT (US); Robert R. Steckel, Norwalk, CT (US); Frederick T. Karl, Bethel, CT (US); Leland R. Adams, Ansonia, CT (US); Robert Chapolini, Phoenix, MD (US)

Correspondence Address: EDWARDS LIFESCIENCES CORPORATION, LEGAL DEPARTMENT, ONE EDWARDS WAY, IRVINE, CA, 92614, US

(21) Appl. No.: 11/186,119

(22) Filed: Jul. 20, 2005

Related U.S. Application Data
(63) Continuation of application No. 09/778,392, filed on Feb. 6, 2001, now abandoned.

(51) Int. Cl. A61B 17/10

(52) U.S. Cl. 606/139

(57) ABSTRACT

The present system is directed to a method and system to stabilize and repair tissue. At least two opposing devices may be used to stabilize and repair the tissue, with the two devices cooperatively engaging the tissue interposed therebetween. Stabilization may be accomplished by opposing force, vacuum force, or mechanical devices disposed at the distal portion of one or both devices. After the tissue has been stabilized, fasteners may be deployed into the tissue. Fasteners include sutures, clips, and staples. Also disclosed is a minimally invasive method of accessing tissue located within a body and conducting a repair of the area using the system disclosed herein.

### RELATED APPLICATIONS

This application is a continuation of U.S. patent application Ser. No. 09/778,392, filed Feb. 6, 2001, entitled “METHOD AND SYSTEM FOR TISSUE REPAIR USING DUAL CATHETERS”, now abandoned

### FIELD OF THE INVENTION

The present invention relates to the repair of tissue, and, more particularly, to a method and apparatus for the repair of tissue within the body of a patient by using a dual catheter system to stabilize the tissue, and if required, fasten the tissue portions together

### BACKGROUND OF THE INVENTION

In vertebrate animals, the heart is a hollow muscular organ having four pumping chambers. The left and right atria and the left and right ventricles, each provided with its own one-way outflow valve. The natural heart valves are identified as the aortic, mitral (or bicuspid), tricuspid and pulmonary valves. The valves separate the chambers of the heart, and are each mounted in an annulus therebetween. The annuluses comprise dense fibrous rings attached either directly or indirectly to the atrial and ventricular muscle fibers. The leaflets are flexible collagenous structures that are attached to and extend inward from the annuluses to meet at coapting edges. The aortic and tricuspid valves have three leaflets, while the mitral and pulmonary valves have two.

Various problems can develop with heart valves, for a number of clinical reasons. Stenosis in heart valves is a condition in which the valves do not open properly. Insufficiency is a condition which a valve does not close properly. Repair or replacement of the aortic or mitral valves are most common because they reside in the left side of the heart where pressures and stresses are the greatest. In a valve replacement operation, the damaged leaflets are excised and the annulus sculpted to receive a replacement prosthetic valve.

In many patients who suffer from valve dysfunction, surgical repair (i.e., “valvuloplasty”) is a desirable alternative to valve replacement. Remodeling of the valve annulus (i.e., “annuloplasty”) is central to many reconstructive valvuloplasty procedures. Remodeling of the valve annulus is typically accomplished by implantation of a prosthetic ring (i.e. “annuloplasty ring”) to stabilize the annulus and to correct or prevent valvular insufficiency that may result from a dysfunction of the valve annulus. Annuloplasty rings are typically constructed of a resilient core covered with a fabric sewing ring. Annuloplasty procedures are performed not only to repair damaged or diseased annuli, but also in conjunction with other procedures, such as leaflet repair.

Mitral valve regurgitation is caused by dysfunction of the mitral valve structure, or direct injury to the mitral valve leaflets. A less than perfect understanding of the disease process leading to mitral valve regurgitation complicates selection of the appropriate repair technique. Though implantation of an annuloplasty ring, typically around the posterior aspect of the mitral valve, has proven successful in a number of cases, shaping the surrounding annulus does not always lead to optimum coaptation of the leaflets.

More recently, a technique known as a “bow-tie” repair has been advocated. The bow-tie technique involves suturing the anterior and posterior leaflets together in the middle, causing blood to flow through the two side openings thus formed. This technique was originally developed by Dr. Ottavio Alfieri, and involved placing the patient on extracorporeal bypass in order to access and suture the mitral valve leaflets.

A method for performing the bow-tie technique without the need for bypass has been proposed by Dr. Mehmet Oz, of Columbia University. The method and a device for performing the method are disclosed in PCT publication WO 99/00059, dated Jan. 7, 1999. In one embodiment, the device consists of a forceps-like grasper device that can be passed through a sealed aperture in the apex of the left ventricle. The two mitral valve leaflets meet and curve into the left ventricular cavity at their mating edges, and are thus easy to grasp from inside the ventricle. The mating leaflet edges are grasped from the ventricular side and held together, and various devices such as staples are utilized to fasten them together. The teeth of the grasper device are linearly slidable with respect to one another so as to align the mitral valve leaflets prior to fastening. As the procedure is done on a beating heart, and the pressures and motions within the left ventricle are severe, the procedure is thus rendered fairly skill-intensive.

There is presently a need for an improved means for performing the bow-tie technique of mitral valve repair, preferably utilizing a minimally invasive technique.

### SUMMARY OF THE INVENTION

The present invention provides a method and system for approximating tissue using at least two catheters. More particularly, the present invention discloses a method and system of approximating a number of devices and methods for stabilizing tissue and fastening or “approximating” a single portion or discrete pieces of tissue through the use of at least two probes directed to the area of interest by at least one guidewire. The tissue of interest may be straight, curved, tubular, etc. For example, many of the embodiments of the invention disclosed herein are especially useful for joining two leaflets of a heart valve. The coapting edges of the leaflets thus constitute the “tissue pieces.” In other contexts, the invention can be used to repair Arterial Septal Defects (ASD), Ventricular Septal Defects (VSD), and in cases involving patent foraman ovale. Additionally, the present invention may be used during valve replacement surgery, to deploy a plurality of valve repair devices. In sum, the present invention in its broadest sense should not be construed to be limited to any particular tissue pieces, although particular examples may be shown and disclosed.

The present invention includes a number of guidewire-directed devices and methods for both stabilizing the tissue pieces to be joined, and fastening them together. Some embodiments disclose only the stabilizing function, others only the fastening function, and still other show combinations of stabilizing and fastening devices. It should be understood that certain of the stabilizing devices may be used with certain of the fastening devices, even though they are not explicitly shown in joint operation. In other words, based on the explanation of the particular device, one of skill in the art should have little trouble combining the features of certain of two such devices. Therefore, it should be understood that many of the stabilizing and fastening devices are interchangeable, and the invention covers all permutations thereof.

Furthermore, many of the fastening devices disclosed herein can be deployed separately from many of the stabilizing devices, and the two can therefore be deployed in parallel.

The guidewire-directed stabilizing and fastening devices of the present invention can be utilized, for example, in endoscopic procedures, beating heart procedures, or percutaneous procedures. In yet another embodiment the devices can be delivered into the heart through the chest via a thorascope. The devices can also be delivered percutaneously, via a catheter or catheters, into the patient's arterial system (e.g. through the femoral or brachial arteries). Other objects, features, and advantages of the present invention will become apparent from a consideration of the following detailed description.

Other objects, features, and advantages of the present invention will become apparent from a consideration of the following detailed description.

### BRIEF DESCRIPTION OF THE DRAWINGS

FIG. 1 is a elevational view of a step in a valve repair procedure using the present invention;

FIG. 1a is an elevational view of an embodiment of a vacuum based probe of the present invention;

FIG. 1b is an elevational view of an embodiment of a vacuum based probe of the present invention disposing including vanes;

FIG. 2 is an elevational view of an embodiment of a vacuum based probe of the present invention having a tapered nose and disposing vanes;

FIG. 2a is an sectional view of a step in a valve repair procedure using the tissue stabilizer of FIG. 2;

FIGS. 3a-3care perspective views of several embodiments of vacuum-based tissue stabilizers having tissue separating walls;

FIGS. 3d and 3e are sectional views of two different vacuum port configurations for the tissue stabilizers shown in FIGS. 3a-3c, the stabilizers shown in operation;

FIG. 4a is an elevational view of a first step in a valve repair procedure using a mechanical tissue stabilizer with linearly displaceable tissue clamps;

FIG. 4b is an elevational view of a second step in a valve repair procedure using the tissue stabilizer of FIG. 4a;

FIG. 4c is a detailed perspective view of a clamp of the tissue stabilizer of FIG. 4a extended to grasp a valve leaflet from both sides;

FIG. 5a is a perspective view of a suture-based tissue fastener of the present invention having toggles;

FIG. 5b is a sectional view of the suture-based tissue fastener of FIG. 5a loaded into a delivery needle;

FIGS. 6a-6c are elevational views of several steps in a valve repair procedure using a tissue stabilizer of the present invention and the suture-based tissue fastener shown in FIG. 5a.

FIG. 7 is an elevational view of an alternative tissue stabilizing and fastening device;

FIGS. 8a-8c are sectional views of a tissue stabilizing and fastening device of the present invention having needles deployed by the retrograde probe on the ventricular side of the tissue being received by the antegrade probe;

FIG. 9a is a perspective of a further tissue fastening device of the present invention comprising a staple-like tissue fastener in an open configuration;

FIG. 9b is a perspective view of further tissue fastening device of the present invention comprising a staple-like tissue fastener in a closed configuration;

FIGS. 10a-10c are sectional views of several steps in a valve repair procedure using an exemplary tissue fastening device of the present invention for delivering the tissue staple of FIGS. 9a-9b;

FIG. 11 is a perspective view of a completed valve repair procedure utilizing the tissue stabilizing and fastening device of FIGS. 10a-10c;

FIG. 12 is an elevational view of an alignment mechanism of the present invention of the present invention;

FIGS. 13a-13b are sectional views of a wire-based steering mechanisms of the present invention;

FIGS. 14a-14b are sectional view of the steering sleeve based steering mechanism of the present invention;

FIG. 15 is a sectional view of the steering balloon based steering mechanism of the present invention; and

FIGS. 16a-16c are sectional views of several steps in a tissue repair procedure using an exemplary sequential tissue repair device of the present.

### DETAILED DESCRIPTION OF THE INVENTION

The method and system of the present invention is designed for use in the surgical treatment of bodily tissue. As those skilled in the art will appreciate, the exemplary guidewire-directed dual catheter tissue repair system disclosed herein is designed to minimize trauma to the patient before, during, and subsequent to the surgical procedure, while providing improved device placement and enhanced tissue stabilization. Additionally, the guidewire-directed dual catheter tissue repair system, by utilizing two separate and distinct probes that cooperatively interact, may be adapted to precisely deliver and deploy a plurality of tissue fasteners to an area of interest. For example, the present system may be utilized to repair mitral valve tissue by stabilizing the discrete tissue pieces and deploying a fastening device thereby coapting the tissue pieces. As those skilled in the art will appreciate, the present invention may similarly used to repair Arterial Septal Defects (ASD), Ventricular Septal Defects (VSD), and defects associated with Patent Foramen Ovale (PFO).

The present invention incorporates by reference many of the device features and various tissue fastening devices disclosed the applicant's pending U.S. application entitled “Minimally Invasive Mitral Valve Repair Method And Apparatus”, application Ser. No. 09/562406 filed May 1, 2000. Disclosed herein is a detailed description of various illustrated embodiments of the invention. This description is not to be taken in a limiting sense, but is made merely for the purpose of illustrating the general principles of the invention. The section titles and overall organization of the present detailed description are for the purpose of convenience only and are not intended to limit the present invention.

As those skilled in the art will appreciate, the present invention permits the operator to position at least two guidewire-directed probes within a body vessel and utilize the cooperative effects of the two positions and deploy a plurality of fastening devices to surrounding tissue. In the illustrated embodiment, the two probes comprise an antegrade probe positioned proximate to the superior or atrial portion of the mitral valve, and a retrograde probe positioned proximate to the inferior or ventricular portion of the mitral valve. It is anticipated as being within the scope of the present invention to utilize the present invention to perform a plurality of surgical procedures, and may deliver and deploy a plurality of tissue fastening devices to an intra-vascular area.

For example, the present device may be utilized to repair defects in the arterial septum. At least two guidewire-directed probes, one probe addressing the tissue from an antegrade position and the other probe addressing the tissue from a retrograde position, are used to stabilize the arterial septal tissue. Once stabilized, a fastening device maybe deployed to repair the defect. Similarly, the present invention maybe used to repair venticular septal defects, or defects relating to patent foramen ovale.

A. Exemplary Procedure Description

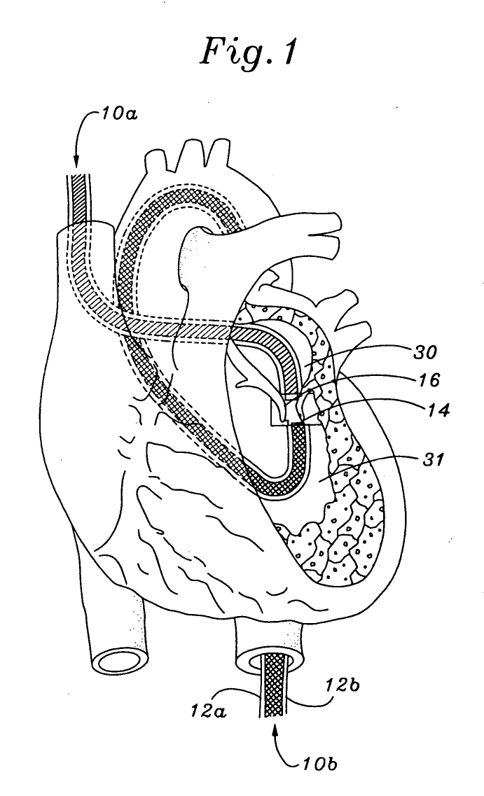

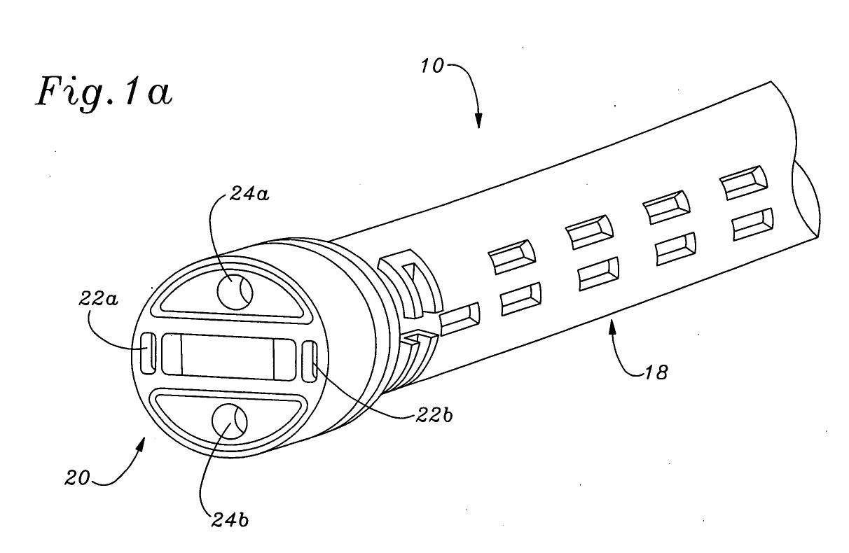

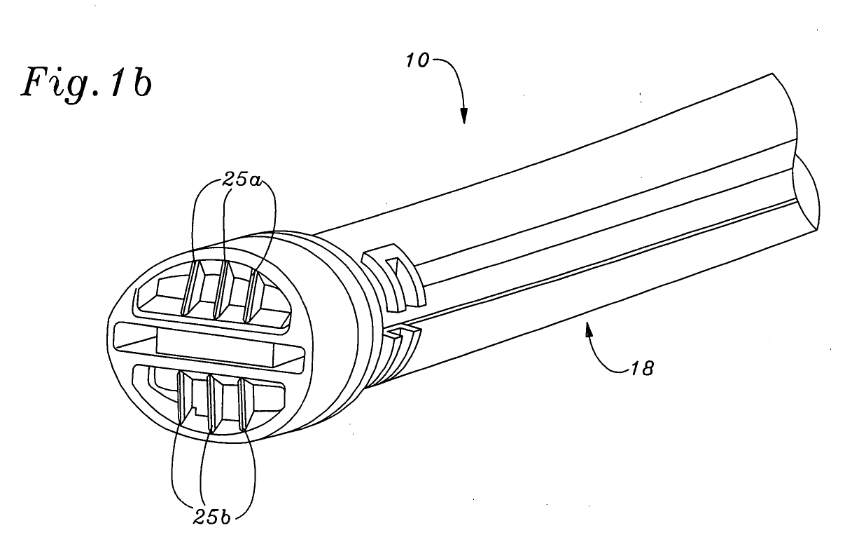

FIG. 1 shows an embodiment of the present invention being utilized to repair a heart valve. More particularly, FIG. 1 shows a guidewire-directed antegrade probe 10a and retrograde probe 10b being used to stabilize and repair the tissue leaflets 14 and 16 of the mitral valve.

A first guidewire 12a, capable of traversing the circulatory system and entering the heart, is introduced into the femoral vein of a patient (or, alternatively the right jugular vein) through an endoluminal entry point. The first guidewire 12a is advanced through the circulatory system eventually arriving at the heart. Upon arriving a the heart, the first guidewire 12a enters the right atrium of the heart. The first guidewire 12a is directed to traverse the right atrium and puncture the atrial septum, thereby entering the left atrium. The first guidewire 12a is progressed through the mitral valve while the heart is in diastole thereby entering into the left ventricle. Thereafter the first guide wire 12a is made to traverse through the aortic valve into the aorta and is made to emerge at the left femoral artery through a endoluminal exit point. This methodology is known to physicians skilled in interventional cardiology. Once first guide wire 12a is positioned, a second guide wire 12b similarly traverses the circulatory system and is positioned proximal to first guide wire 12a using techniques familiar to those skilled in the art. The endoluminal entry and exit ports are dilated to permit entry of at least one probe. A protective sheath may be advanced within the venous area to protect the inner venular structure.

With guidewires 12a and 12b suitably anchored, the antegrade probe 10a is attached to the guidewires 12a and 12b and advanced through the dilated guide wire entry point to a point proximal to the arterial cusp portion of the mitral valve. The distal portion of antegrade probe 10a, having at least one vacuum port in communication with at least one vacuum lumen contained within at least one internal lumen of the probe, is positioned proximate the tissue leaflets 14 and 16 of the mitral valve. Once positioned, the antegrade probe 10a may use vacuum force to capture and grasp the mitral tissue, grasp the tissue and deploy a fastening device, grasp and manipulate the mitral tissue, or grasp and manipulate the tissue to a desired positioned and deploy a fastening device. The manipulation or steering of the mitral tissue is accomplished by positioning the at least one vacuum port proximate the mitral tissue and activating the vacuum source. The mitral tissue will be forcibly retained by the vacuum force, thereby permitting the operator to steer or position tissue.

A retrograde probe 10b is attached to at least one guidewire and introduced into the body through dilated guidewire exit point. The flexible retrograde probe 10b is advanced through the body vessel, entering the heart through the aortic valve and progressing into the left ventricle. The distal portion of retrograde probe 10b is proximal the ventricular portion of the of the mitral valve. The retrograde probe 10b may include a distal portion having at least one vacuum port connected to at least vacuum lumen contained within at least one internal lumen, thereby permitting retrograde stabilization of tissue.

With the antegrade probe and retrograde probe suitably positioned, the external vacuum source connected to the antegrade probe, retrograde probe, or both, is activated, thereby permitting mechanical capture of the tissue. Upon successful tissue capture, a detachable fastening device mechanically retained either by antegrade probe 10a or retrograde probe 10b, or both, is forcibly deployed piercing the valve tissue and thereby mechanically joining the cusps of the mitral valve. These fastening devices may include self-closing fasteners, spring loaded fasteners, pre-formed fasteners, latching fasteners, and rotatably deployed fasteners.

To complete the procedure, the external vacuum source is deactivated, resulting in tissue release. The two probes are retracted through their individual entry points, and the two guidewires are removed. Finally, the endoluminary entry point and exit point are sutured.

B. Exemplary Guidewire Devices

FIG. 1 shows a guidewire-directed dual catheter tissue stabilizer system comprising an antegrade probe 10a and a retrograde probe 10b of the present invention that is used to stabilize two tissue pieces 14 and 16, respectively. The guidewires 12a and 12b may be formed of a single filament or a multi-filament wound system, and may be comprised of materials known to those skilled in the art of minimally invasive surgery, including, without limitation, a Nickel-Titanium (Ni Ti) compound, stainless steel #304, 304V, 312, and 316, or other suitable material. Likewise, the guidewires may be coated with a biologically-compatible lubricant or with a biologically-compatible sealant such as polytetrafluoroethylene (PTFE). The guidewires should have sufficient structural flexibility and steerability to permit intraluminal positioning, while retaining sufficient structural integrity to position tissue stabilizers. Additionally, the guidewires may have a substantially circular profile, or, alternatively, may be shaped to provide a degree of axial control. For example, a wire incorporating a substantially octagonal profile would provide sufficient axial force to permit axial movement of the catheters along an axial arc.

During a procedure, a guidewire 12a may be introduced to a body vessel in a plurality of manners, including, for example and without limitation, percutaneously, transapically, transatrially, or through a surgical incision proximate the area of interest. Guidewire 12a is then positioned proximate to or traversing the area of interest. Once positioned and sufficiently anchored, a second guidewire 12b may be similarly introduced to traverse the pathway established by guidewire 12a, and likewise positioned within the mitral valve and suitably anchored. It should be understood that the present invention contemplates without limitation either a single guidewire or multiple guidewire approach. These guidwire or guidewires will direct and precisely position probes 10a and 10b proximate the area of interest. Upon completion of the procedure, the probes 10a and 10b and the guidewire (not shown) or guidewires 12a and 12b are removed from the body vessel.

C. Exemplary Tissue Stabilizing Devices

It should be understood that the antegrade and the retrograde probe disclosed herein cooperatively interact to provide stabilizing force to the tissue interposed therebetween. For example, the cooperative interaction may consist of the application of force to opposing surfaces of tissue interposed between the probes, vacuum force applied by either or both probes, and mechanical retaining devices, as detailed below, disposed on either or both probes. It is understood that both probes utilize at least one guidewire slidably attached to the distal portion of each probe to precisely position and align the probes. Furthermore, it is understood that the antegrade probe or the retrograde probe, or both, may apply the retentive force to stabilize tissue. Additionally, tissue fastening device may be disposed about the proximal portion of the antegrade probe or the retrograde probe, or both, to approximate two pieces of tissue disposed between the opposing probes. A deployable alignment mechanism may be disposed about the distal portion of the antegrade probe or retrograde probe, or both, thereby ensuring a precise positioning of either or both probes with relation to the tissue.

FIG. 1 shows two probes 10a and 10b of the present invention that uses a vacuum to stabilize two tissue pieces 14 and 16, respectively. In this case, the procedure being conducted is a repair of a heart valve using an arterial probe 10a and a ventricular probe 10b. The at least two probes 10a and 10b may share common elements and will be generically described as probe 10.

As shown in FIG. 1a, the probe 10 comprises a cylindrical probe body 18 with at least one internal lumen (not shown) and having a flat distal portion 20 disposing at least two guidewire ports, 22a and 22b, and at least two vacuum ports 24a and 24b. It should be noted that the illustrated embodiment utilizes two guidwires, though the system may be operated using a single guidewire. The at least two guidewire ports, 22a and 22b, which are connected to at least two guidewire lumens (not shown), are disposed radially about the distal portion 20 of the probe 10, and are substantially parallel to the longitudinal axis of at least one internal lumen (not shown). The at least two vacuum ports 24a and 24b, are in communication with an external vacuum source through the at least one internal lumen (not shown). The size of the ports, namely 24a and 24b, and magnitude of suction applied may be vary depending on the application. The spacing between the ports 24a and 24b should be sufficiently spaced so as to create independent suction regions. In this manner, one leaflet or the other may be stabilized with one of the ports, e.g. 24a, without unduly influencing the other port, e.g. 24b. In one example, the ports 24a and 24b have a minimum diameter of about ⅛ inch, and are spaced apart with a wall of at least 0.020 inches therebetween.

As shown in FIG. 1b, the distal portion 20 may dispose a series of vanes, 25a and 25b, positioned proximate the vacuum ports 24a and 24b. The vane series, 25a and 25b, respectively, may be recessed from the distal portion 20, thereby forming a tissue supporting structure when vacuum force is applied to pliable tissue. Preferably, the vanes 25a and 25b are recessed approximately 0.002 to 0.01 inches from the distal portion 20.

The probe 10 desirably has a size suitable for minimally invasive surgery. In one embodiment probe 10 is part of a catheter based percutaneous delivery system. In that case probe 10 is a catheter tube having one or more lumens connecting vacuum ports 29a and 29b to the vacuum source or sources. The catheter would be long enough and have sufficient steerability and maneuverability to reach the heart valve from a peripheral insertion site, such as the femoral or brachial artery. One particular advantage of the present invention is the ability to perform valve repair surgery on a beating heart.

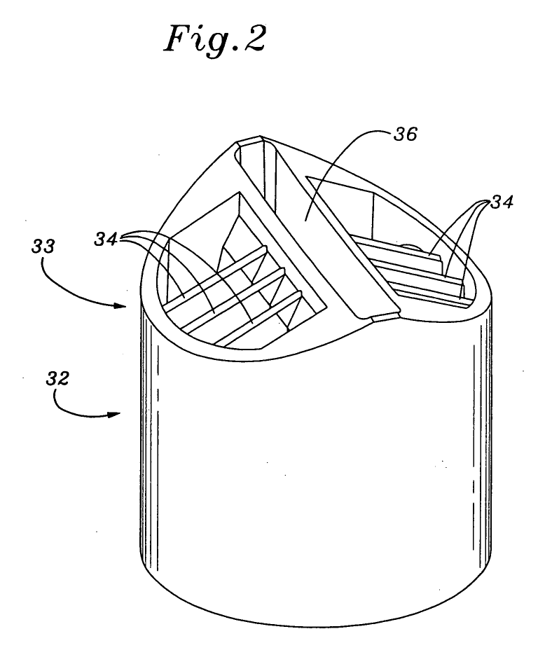

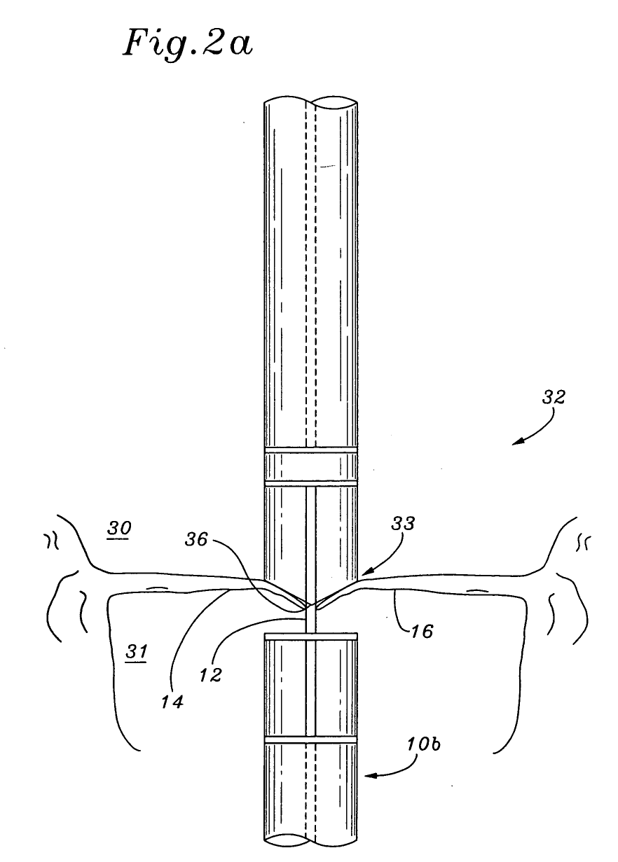

FIG. 2 illustrates an additional embodiment of the present invention utilizing a tapered distal portion of the probe. The probe 32 has a distal portion which includes a series of recessed vanes 34 connected to at least one internal lumen (not shown) to stabilize tissue. An additional port 36 may be used to deploy or receive a plurality of fastening devices.

FIG. 2a shows an illustrative valve repair procedure using the probe 32 of FIG. 2 approaching the tissue from the arterial side 30 of the valve, while additionally stabilizing the tissue with probe 10b from the ventricular side 31 of the valve. The port 36 at the distal tip of the nose is exposed to the ventricular 31 side of the leaflets 14 and 16. Because of this exposure, various leaflet fastening devices can be delivered through the probe 32 to the ventricular 31 side of the leaflets 14 and 16, as will be detailed below. Likewise, a tissue fastening device may be deployed by probe 10b through the leaflets, 14 and 16, to the probe 32 positioned proximal to the arterial portion of the mitral valve. Interference with the stabilization process by guidewire 12 is negligible. Those skilled in the art will appreciate either the antegrade probe, the retrograde probe, or both, may utilize the tapered nose design detailed herein.

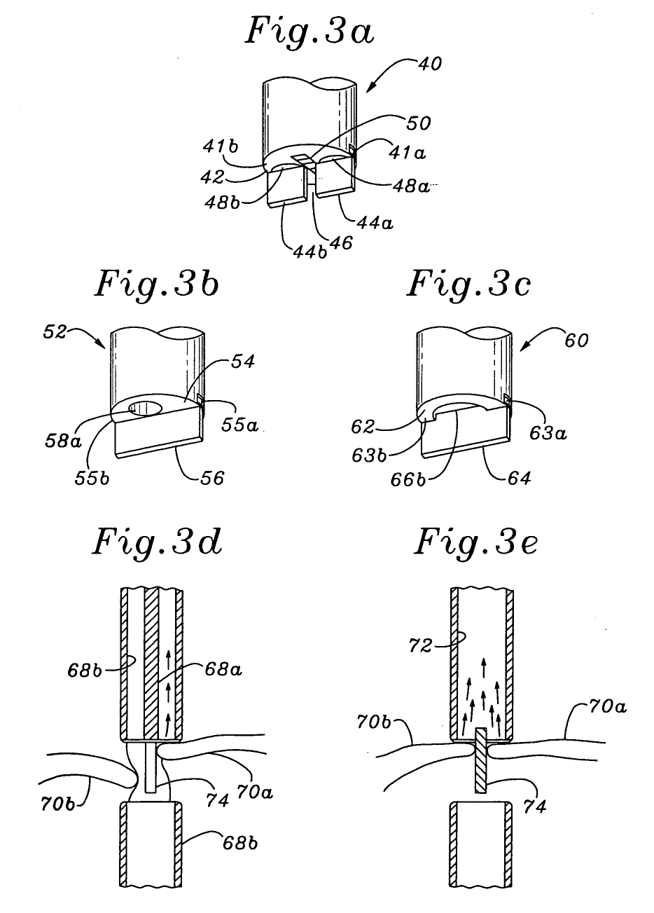

FIGS. 3a-3c show three vacuum-based tissue stabilizing probes having tissue separating walls. In FIG. 3a, a tissue stabilizer 40 includes at least two guidewire ports 41a and 41b radially about the distal portion of the probe, having a flat distal face 42 having a pair of distally-directed tissue separating walls 44a and 44b extending therefrom, and defining a gap 46 therebetween. The stabilizer 40 contains one or more lumens in communication with vacuum ports 48a and 48b, that open on both sides of the walls 44a and 44b. In addition, a fastener channel 50 opens at the distal face 42 between the walls 44a and 44b, and facing the gap 46 therebetween. The fastener channel 50 can be used to deliver tissue fasteners, as described below.

In FIG. 3b, a tissue stabilizer 52 includes a flat distal face 54 disposing at least two guidewire ports 55a and 55b, and having a single distally-directed tissue separating wall 56 extending therefrom. The stabilizer 52 contains one or more lumens in communication with circular vacuum ports 58a and 58b (not shown) that open on either side of the wall 56.

In FIG. 3c, a tissue stabilizer 60 includes a flat distal face 62, disposing at least two guidewire ports 63a and 63b radially positioned about distal face 62, and having a single distally-directed tissue separating wall 64 extending therefrom. The stabilizer 60 contains one or more lumens in communication with semi-circular vacuum ports 66a (not shown) and 66b that open on both sides of the wall 64. There are two such ports 66a (not shown) and 66b, one on each side of each wall 64.

FIGS. 3d and 3e show two different vacuum port configurations for the tissue stabilizers 40, 52, or 60 shown in FIGS. 3a-3c. As mentioned above, the stabilizers 40, 52, or 60 may have one or more lumens in communication with one or more ports. In FIG. 3d, two lumens 68a and 68b provide separate suction control to the associated ports. Thus, one tissue piece 70a is seen stabilized by the right-hand vacuum port, while the left-hand port is not operated. Alternatively, a single lumen 72 in communication with two vacuum ports is seen in FIG. 3e, and both tissue pieces 70a, 70b are stabilized simultaneously. In both these views, the tissue separating wall 74 is shown between the tissue pieces to be joined. Fastening devices can thus be delivered via the wall 74, or through a gap formed for that purpose, such as the gap 46 and fastener channel 50 seen in FIG. 3a.

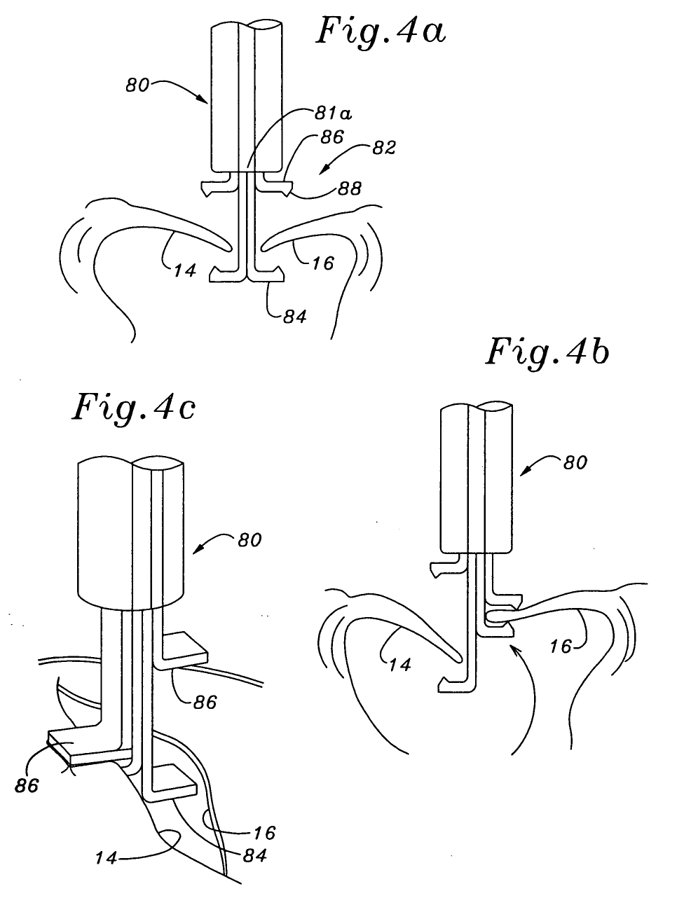

FIGS. 4a-4c show a mechanical tissue stabilizer 80 with a four-part, linearly displaceable tissue clamp 82, disposing at least two guidewire ports 81a and 81b (not shown), respectively, positioned radially about the distal portion of the stabilizer 80. On each side, a lower clamp 84 is separated from an upper clamp 86 and inserted between two tissue pieces (in this case valve leaflets 14 and 16). As the lower and upper clamps 84, 86 are brought together, as seen in FIG. 4b, they physically clamp and stabilize the leaflet 16. Small teeth 88 on the clamps 84 and 86 may be provided for traction. The clamps 84 and 86 on each side are individually actuated to enable grasping of one leaflet at a time. Once the tissue has been suitably captured by antegrade probe 80 an retrograde probe (not shown) is utilized to deploy a fastening device to the captured tissue.

As stated above, the dual catheter system disclosed herein contemplates utilizing the probes disclosed above in a cooperative manner. As those skilled in the art will appreciate, various arterial probes may be used with various ventricular probes, thereby providing a dual catheter system capable of customization dependant on need. For example, an arterial probe having a tapered nose may be used with a ventricular probe having a flat distal portion. Alternatively, an arterial probe having a flat distal portion may be utilized with a ventricular probe having a tapered nose. As those skilled in the art will appreciate the system may be easily tailored accordingly.

D. Exemplary Tissue Fasteners

As stated in the previous sections, the present invention contemplates using at least one guide wire to direct and position at least two co-operatively functioning probes to an area of interest. In a preferred embodiment, at least two probes, each disposing at least two guidewire ports proximate to the distal portion thereof, would be directed to an area of interest by at least two guidewires. It should be understood that the present invention discloses using at least two guidewire-directed probes simultaneously to perform a surgical therapeutic procedure. The following sections disclose exemplary tissue fasteners capable of deployment with the guidewire-directed dual catheter system of the present invention. The figures associated with the following sections are intended to illustrate novel fastening systems. As such, only one catheter may be illustrated, but a second catheter is assumed. Likewise, the following systems employ at least one guidewire and at least two guidewire ports disposed proximal the distal portion of the probes. To permit clear illustration of the novel fastening systems disclosed herein the guidewire or guidewire and guidewire ports may not be illustrated in the following figures, but should be assumed included.

1. Exemplary Suture-Based Tissue Fasteners

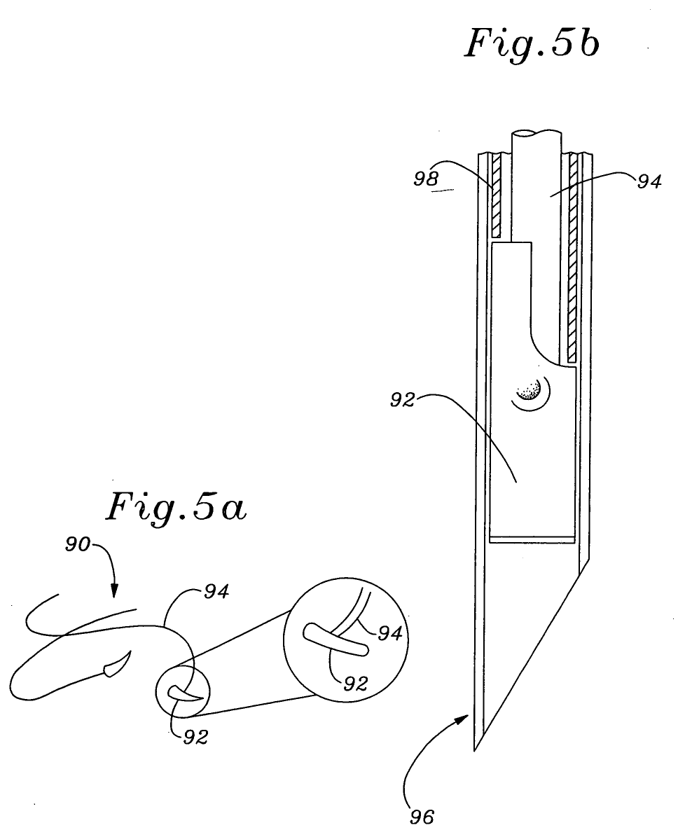

FIG. 5a illustrates a suture-based tissue fastener 90 of the present invention including toggles 92 secured to the end of suture threads 94. FIG. 5b is a sectional view through a needle 96 used to deliver the tissue fastener 90. Specifically, the toggle 92 and suture thread 94 is seen loaded into the lumen of the needle 96, and a pusher 98 is provided to urge the tissue fastener 90 from the distal end thereof. The fastener 90 maybe deployed by the antegrade probe, the retrograde probe, or both.

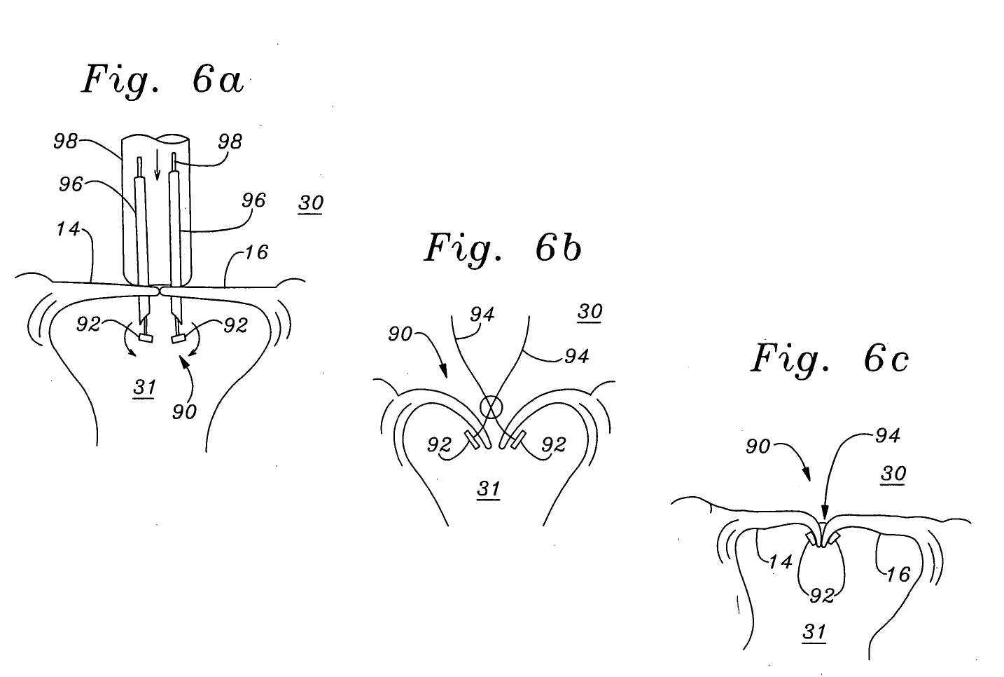

FIGS. 6a-6c depict several steps in a valve repair procedure using the tissue fasteners 90 shown in FIG. 5a. A probe, such as the probe 10 seen in FIG. 1 having vacuum ports for tissue stabilization and guidewire ports positioned radially about the distal portion of probe 10, provides lumens for two of the needles 96 of FIG. 5b. The lumens with the vacuum ports may receive the needles 96 or additional lumens may be provided. The sharp ends of the needles 96 pierce the leaflets, and the pushers 98 are displaced (separately or in conjunction) to deploy the tissue fasteners 90. After the needles 96 are retracted, the toggles 92 anchor the tissue fasteners 90 on the ventricular 31 side of the leaflets. The suture threads 94 are then tied off on the atrial 30 side to secure the leaflets 14 and 16 together, as seen in FIG. 6c. The retrograde probe used to stabilize the tissue is not shown to permit clear illustration of the novel fastening device. As with all system disclosed herein, simultaneous use of an antegrade probe and retrograde probe is contemplated.

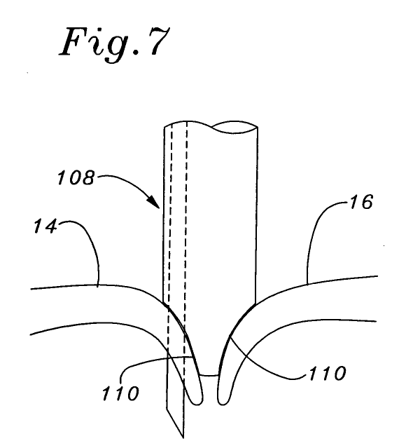

FIG. 7 illustrates an alternative tissue stabilizing and fastening device 108 having a pointed nose with two concave faces 110 in which the vacuum ports are located. The device 108 functions as described above, with a fastener deliver needle shown in phantom having pierced the left leaflet 14. A retrograde probe (not shown) may be adapted to receive the fastening device 108 as well as stabilize the tissue.

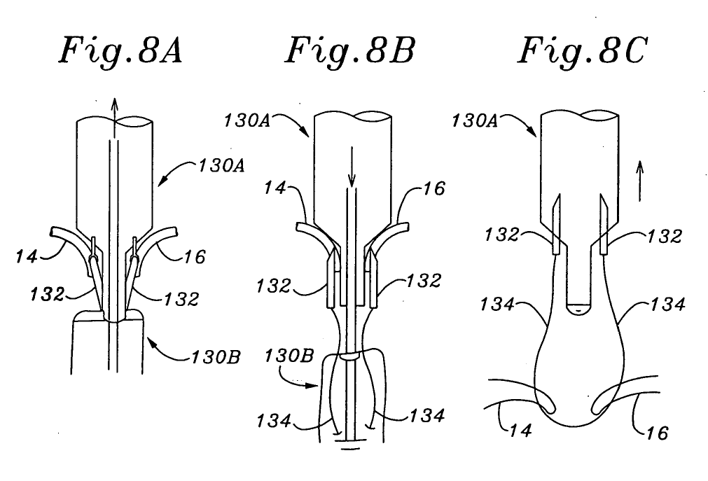

FIGS. 8a-8c illustrate a tissue stabilizing and fastening device 130a-b having needles 132 deployable on a blind side of the tissue by the retrograde probe 130b. A common suture thread 134 connects the needles 132 and is used to secure the tissue pieces 14 and 16 together. Thus, as seen in the sequence of FIGS. 8a-8c, the needles 132 are first advanced to a position proximate the tissue pieces 14 and 16 and deployed outboard of the distal tip of the retrograde probe 130b. Once positioned, the needles are advanced through the tissue, as in FIG. 8a, to cause the needles 132 to pierce the tissue pieces 14 and 16. The two needles 132 are then disengaged from the device 130b, and each other, as in FIG. 8b, and antegrade probe 130a captures the needles 132 from the pieces 14 and 16, leaving the connected suture joining the two pieces 14 and 16 (FIG. 8c). The suture 132 can then be tied off, or otherwise secured on the upper side of the tissue pieces 14 and 16.

2. Exemplary Staple and Clip-Type Fasteners

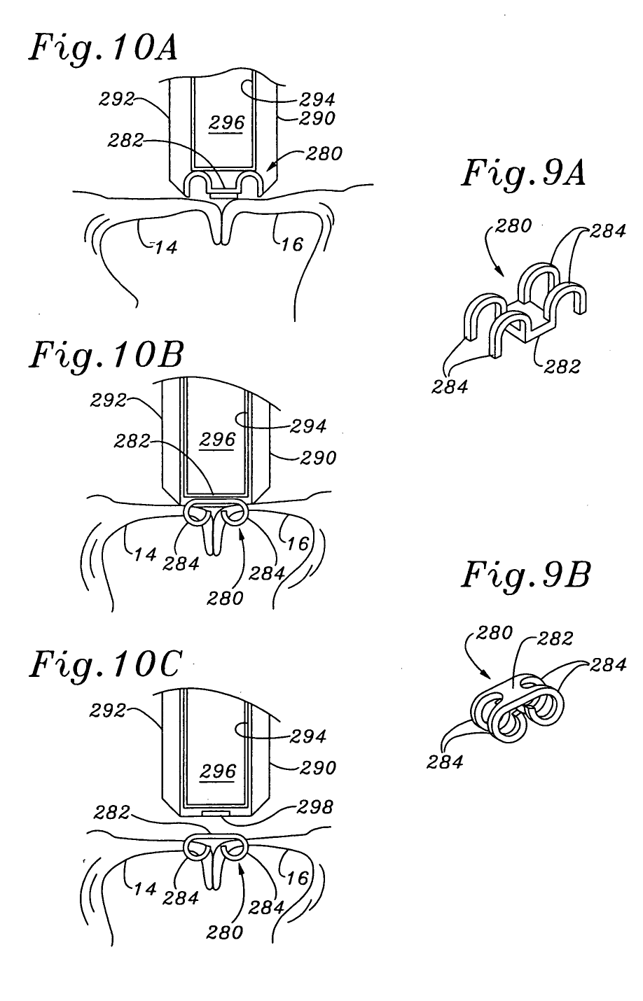

FIG. 9a shows an exemplary tissue staple 280 for joining two tissue pieces in an open configuration. The staple 280 includes a bridge portion 282 and four gripping arms 284, two on each side. The gripping arms 284 are initially curled in a semi-circle upward from the plane of the bridge portion 282 and terminate in sharp points approximately in the plane of the bridge portion 282. FIG. 9b shows the staple 280 when closed, with the gripping arms 284 curled underneath the plane of the bridge portion 282 toward each other.

FIGS. 10a-10c illustrate several steps in a valve repair procedure using an exemplary tissue fastening device 290 for delivering the tissue staple 280. As with the previous embodiments, a retrograde probe (not shown) is utilized to stabilize the tissue prior to and during deployment of the fastening device. Additionally, the retrograde probe (not shown) may be used as an anvil or stop-body to assist in closing the fastener. The device 290 includes a probe 292 with an internal lumen 294 within which a pusher 296 is slidable, and having at least two guidewire ports (not shown) positioned radially about the distal portion of the probe. A stop member 298 is also provided underneath the bridge portion 282 of the staple 280 to prevent displacement of the bridge portion 282 toward the leaflets 14 and 16. The probe is positioned proximate the tissue under repair. After stabilizing the leaflets 14 and 16, the pusher 296 displaces downward which causes the staple 280 to undergo a plastic deformation from the configuration of FIG. 10a to that of FIG. 10b. The sharp points of the gripping arms 284 pass through the leaflets 14 and 16 and anchor the staple 280 therein. Finally, the stop member 298 is disengaged from under the bridge portion 282, and the device 290 is retracted.

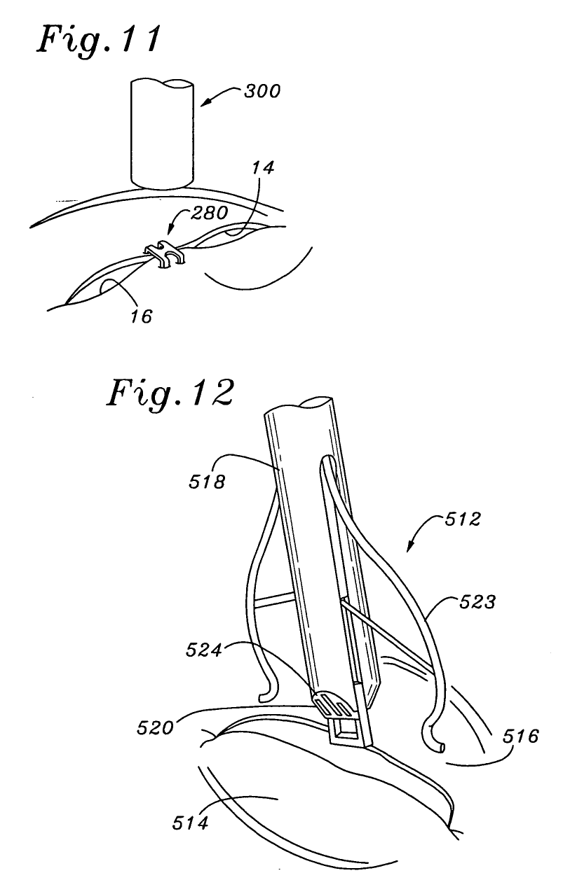

FIG. 11 illustrates the use of a tissue stabilizing and fastening device 300 for deploying the staple 280 of FIG. 9. The device 300 is quite similar to the device 290 of FIG. 10, with an exemplary stabilizing means shown in the form of vacuum chamber(s) 302 on each side of the staple deployment mechanism.

The present invention may be embodied in other specific forms without departing from its spirit, and the described embodiments are to be considered in all respects only as illustrative and not restrictive. The scope of the invention is, therefore, indicated by the claims and their equivalents rather than by the foregoing description.

E. Exemplary Probe Alignment Devices

An additional embodiment of the present invention includes alignment mechanisms which may be affixed to the probe to precisely position a probe proximate within a body vessel. Those skilled in the art will appreciate the use of an alignment device in addition to the guidewire or guidewires disclosed above provides an inherently redundant alignment scheme, thereby permitting a more precise positioning of the probe relative to the area of interest.

FIG. 12 shows an antegrade probe of the antegrade and retrograde probe system of the present invention that uses a vacuum to hold two tissue pieces 514 and 516, respectively. In this case, the tissue pieces are heart valve leaflets, 514 and 516, and a valve repair procedure using an arterial probe 512a and a ventricular probe 512b (not shown). Probes 512a and 512b will hereinafter be generically described as probe 512. As shown in FIG. 12, the probe 512 comprises a cylindrical probe body 518 with at least one internal lumen (not shown) and having a tapered distal portion 520 disposing at least one guidewire port (not shown) and at least one vacuum port 524. At least one deployable alignment mechanism 523 is positioned proximate the probe distal portion 520 and is in communication with the handpiece (not shown) by a deployment conduit (not shown) positioned in at least one internal lumen (not shown) contained within probe 512. Once the probe 512 is positioned proximate to the tissue 514 and 516, respectively, the deployable alignment mechanism 523 is deployed and interacts with the surrounding tissue. The external vacuum source (not shown) is then activated. The at least one vacuum port 524 stabilizes tissue pieces 514 and 516. Upon completion of the procedure, the deployable alignment mechanism 523 is retracted to facilitate removal of the probe 512. While FIG. 12 shows the deployable alignment mechanism disposed on an antegrade probe, either the antegrade probe, retrograde probe, or both, may include deployable alignment devices.

F. Exemplary Steering Devices

The present invention discloses a guidewire-directed system for repairing body tissue. Use of guidewire-directed flexible antegrade and retrograde catheters permits positioning of the devices proximal the tissue under repair. Locating the device proximate tissue under repair may be facilitated by supplemental steering mechanisms capable of permitting the probes to traverse acute angles. Several embodiments detailing a plurality of steering mechanisms are disclosed herein. The steering devices disclosed herein permit positioning of the antegrade catheter, retrograde catheter, or both, should supplemental steering mechanisms be required.

1. Steering Wire Approach

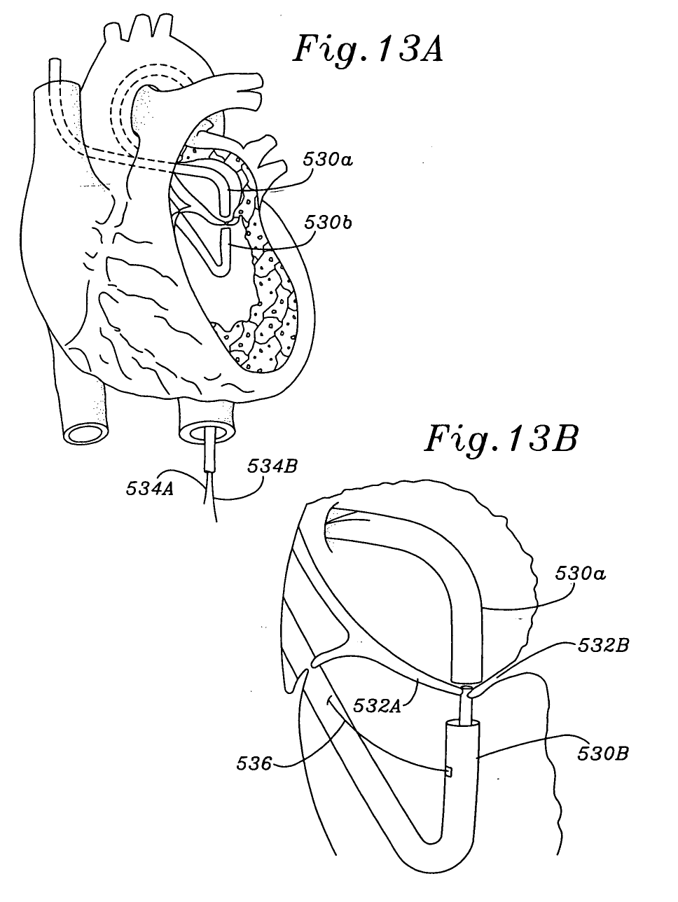

FIGS. 13a-13b show a mitral valve procedure being performed with the present invention. Antegrade probe 530a is positioned proximate the arterial portion of the mitral tissue 532a and 532b by guidewires 534a and 534b. The retrograde probe 530b is positioned proximate the ventricular portion of the mitral tissue 532a and 532b, and is similarly directed by guidewires 534a and 534b. Retrograde probe 530b further disposes a steering conduit 536 which is connected to probe 530b proximate the distal portion and which is in communication with the operator via at least one internal lumen (not shown) through a steering conduit port positioned on probe 530b. The steering conduit 536 may be manufactured from a plurality of materials including a Nickel-Titanium (Ni Ti) compound, stainless steel #304, 304V, 312, and 316, or other suitable material.

2. Steering Sleeve Approach

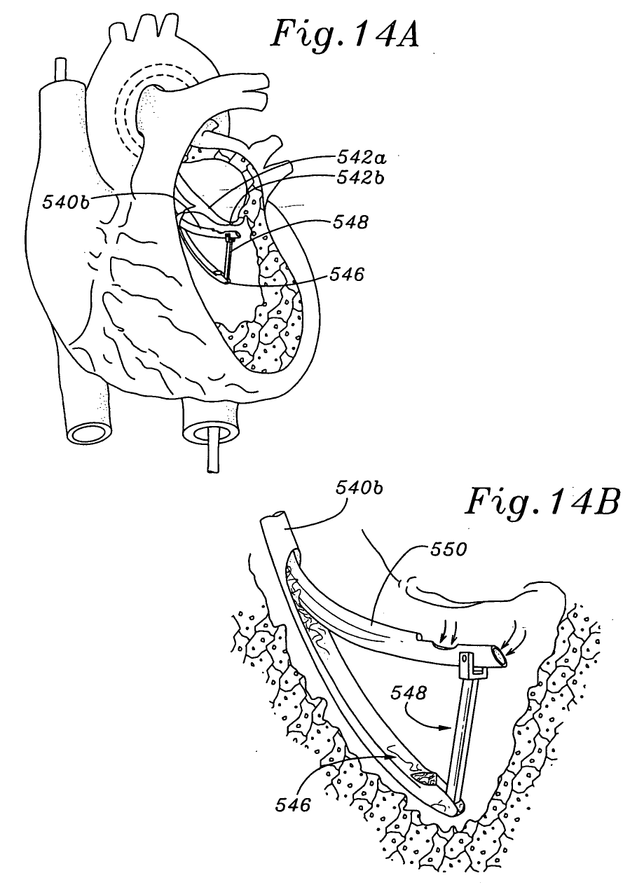

FIGS. 14a-14b show a mitral valve procedure being performed by the present invention. Antegrade probe (not shown) is positioned proximate the arterial portion of the mitral tissue 542a and 542b by guidewires (not shown). The retrograde probe 540b is positioned proximate the ventricular portion of the mitral tissue 542a and 542b, and is similarly directed by the guidewires. Retrograde probe 540b further disposes a steering sleeve 546 containing an actuated support 548 which is connected to a steering sleeve conduit 550 which is positioned within an internal lumen located in the probe 540b. The probe 540b and steering sleeve conduit 550 are positioned proximate the tissue under repair. Once positioned, probe 540b is advanced while the steering sleeve 546 is held stationary. Advancement of the probe 540b results in extension of the actuated support 548 thereby positioning probe 540b more proximate the tissue under repair.

3. Steering Balloon Approach

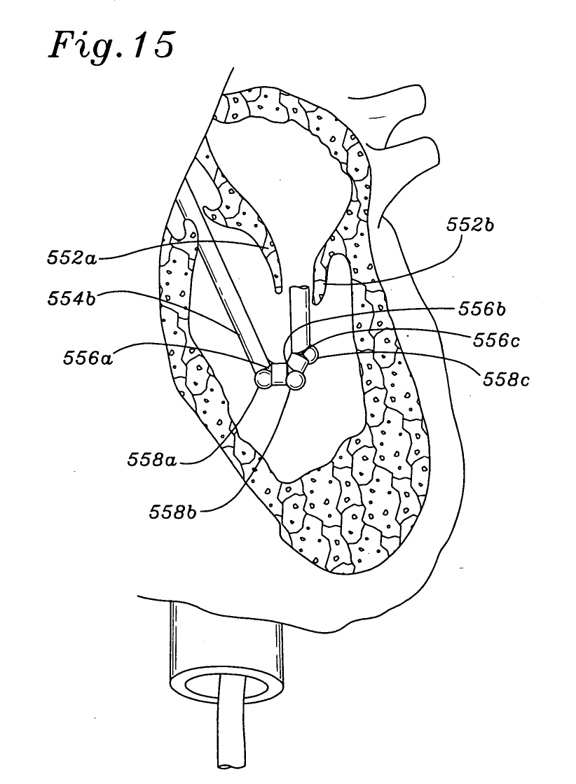

FIG. 15 shows a mitral valve procedure being performed by the present invention. Antegrade probe (not shown) is positioned proximate the arterial portion of the mitral tissue 552a and 552b by guidewires (not shown). The retrograde probe 554b is positioned proximate the ventricular portion of the mitral tissue 552a and 552b, and is similarly directed by the guidewires. Retrograde probe 554b further disposes at least one biasing joint containing at least one balloon which is connected to an inflation conduit (not shown) positioned within an internal lumen located in the probe 554b. FIG. 15 shows a probe 554b disposing 3 biasing joints 556a, 556b, and 556c, each containing a steering balloon 558a, 558b, and 558c, respectively. The probe 554b is positioned proximate the tissue under repair. Once positioned, steering balloons 558a, 558b, and 558c are inflated thereby articulating the distal portion of the probe 554b at an angle proximate the tissue.

G. Sequential Tissue Stabilization

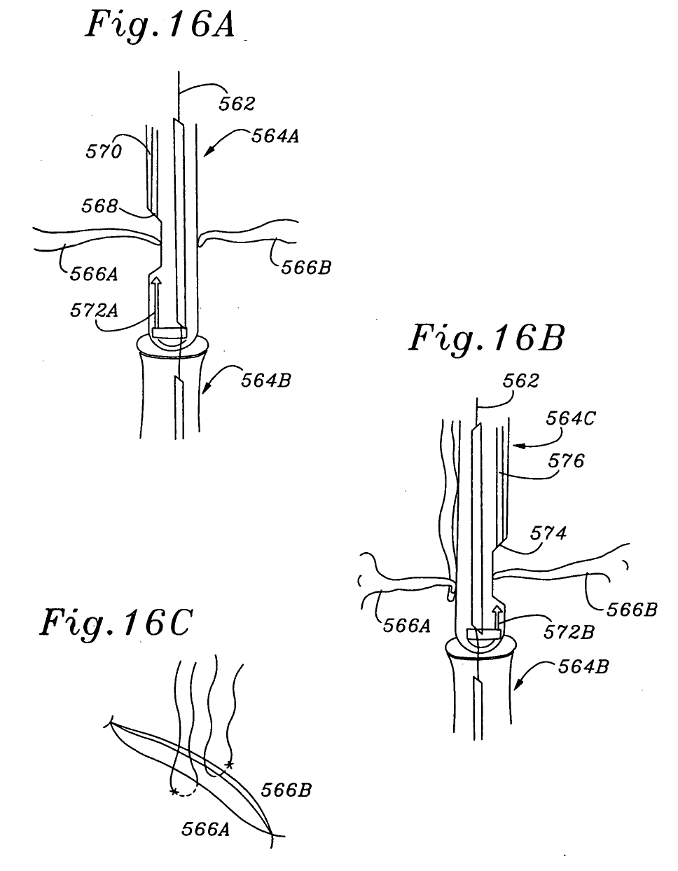

The present invention may be adapted to sequentially stabilize a portion of tissue and deploy a tissue fastening device therein. As shown in FIG. 16a, a first antegrade probe 564a is advanced along at least one guidewire 562 to a position proximate the tissue to be repaired 566a and 566b. The first antegrade probe 564a comprises a vacuum port 568 in fluid communication with a vacuum lumen 570 and a tissue fastening device 572a located within the probe 564a. The tissue fastening device 572a may include fastener deployment mechanisms and fasteners disclosed above. A retrograde probe 564b, which is used to position and stabilize the antegrade probe, is advanced along the at least one guidewire 562 to a position proximate the retrograde portion of the tissue. With the probes 564a and 564b positioned, a single portion of tissue 566a is captured by the vacuum port 568 disposed on the first antegrade probe 564a. A fastening device 572a is deployed through the single portion of tissue 566a. The first antegrade probe 564a disengages the tissue 566a and the retrograde probe 564b, and is thereafter removed. FIG. 16b shows a second antegrade probe 564c comprising a vacuum port 574 in fluid communication with a vacuum lumen 576, and a tissue fastening device 572b located within the probe 564c is advanced to a position proximate the tissue 566a and 566b. Like the first antegrade probe 564a, the second antegrade probe 564c is adapted to engage the retrograde probe 564b, and deploy a tissue fastener. Once the probes are positioned, the vacuum port 574 disposed on the second antegrade probe 564c captures tissue portion 566b. A tissue fastener 572b is deployed into the tissue. The second antegrade probe 564c disengages the tissue 566b, and the second antegrade probe 564c and retrograde probe 564b are removed. As shown in FIG. 16c, the tissue fastening device is joined, for example, by tying, thereby repairing the tissue. Like the previous embodiments the probes 564a, 564b, and 564c may include additional internal lumens.

In closing, it is noted that specific illustrative embodiments of the invention have been disclosed hereinabove. However, it is to be understood that the invention is not limited to these specific embodiments. Accordingly, the invention is not limited to the precise embodiments described in detail hereinabove. With respect to the claims, it is applicant's intention that the claims not be interpreted in accordance with the sixth paragraph of 35 U.S.C. § 112 unless the term “means” is used followed by a functional statement. Further, with respect to the claims, it should be understood that any of the claims described below can be combined for the purposes of the invention.

1. A device for performing a surgical procedure on a heart valve, comprising:
a probe having a distal portion configured for placement within a heart valve annulus, said probe distal portion comprising at least one deployable alignment mechanism.

2. The device of claim 1, wherein the deployable alignment mechanism comprises at least two alignment arms movably attached to the distal portion of the probe.

3. The device of claim 2, wherein each of the alignment arms is configured to interact with the tissue of the heart valve.

4. The device of claim 2, wherein the first alignment arm is positioned on a first side of the probe distal end, and the second alignment arm is positioned on a second side of the probe distal end, wherein the first side of the probe is opposite to the second side of the probe.

5. The device of claim 2, wherein the deployable alignment mechanism further comprises:
a deployment conduit operably connected to said at least two alignment arms;
a deployment actuator attached to the deployment conduit;
said at least two alignment arms having a retracted position wherein said arms are located within the distal portion of the probe;
said at least two alignment arms having a deployed position wherein said arms are extended radially from the distal portion of the probe; and
wherein said retracted and deployed positions are achieved through manipulation of said deployment actuator.

6. The device of claim 1, wherein the probe further comprises:
a first vacuum lumen; and
a first vacuum port at a distal end of the first vacuum lumen, wherein the first vacuum port is configured to grasp heart valve leaflet tissue when a vacuum is applied to the first vacuum lumen.

7. The device of claim 6, wherein the probe further comprises:
a second vacuum lumen; and
a fsecond vacuum port at a distal end of the second vacuum lumen, wherein the second vacuum port is configured to grasp heart valve leaflet tissue when a vacuum is applied to the second vacuum lumen.

8. The system of claim 1, wherein the probe has sufficient length, steerability and maneuverability to reach the heart valve from a peripheral insertion site.

9. A system for performing a surgical procedure on a heart valve, comprising:
at least one guidewire, the guidewire inserted into the heart valve via a blood vessel;
a probe having a first lumen configured to accommodate the guidewire, and a second lumen, the probe having a distal end portion configured for placement within a heart valve annulus, said probe distal portion comprising at least one deployable alignment mechanism.

10. The system of claim 9, wherein the second lumen comprises an alignment mechanism deployment lumen.

11. The system of claim 9, wherein the probe distal portion further comprises at least one tissue fastener.

12. The system of claim 11, wherein the tissue fastener is a suture-based tissue fastener.

13. The system of claim 11, wherein the second lumen comprises a tissue fastening lumen.

14. The system of claim 9, wherein the second lumen comprises a vacuum lumen, and the probe further comprises a vacuum port at the distal end of the vacuum lumen, wherein the vacuum port is configured to grasp heart valve tissue when a vacuum is applied to the vacuum lumen.

15. A method of stabilizing leaflet tissue in a heart valve, comprising:
delivering a probe to a position adjacent the leaflet tissue and heart valve;
aligning the probe with a desired location adjacent the heart valve by deploying one or more radially-deployable alignment mechanisms from the probe, wherein aligning the probe further includes engaging the one or more redially-deployable alignment mechanisms with tissue adjacent the leaflet tissue;
stabilizing the leaflet tissue with one or more stabilizing devices on the probe; and
fastening the leaflet tissue with one or more tissue fasteners.

16. The method of claim 15, wherein the radially-deployable alignment mechanisms comprise radially-deployable alignment arms configured to be positioned within the probe in a first position and configured to radially extend from the probe in a second position, and wherein aligning the probe comprises radially extending the alignment arms from the probe.

17. The method of claim 16, wherein the heart valve is a mitral valve having a first leaflet and a second leaflet, wherein the first leaflet and the second leaflet define a generally elongated heart valve opening therebetween, with the generally elongated heart valve opening comprising a first end and a second end, and wherein the radially-deployable alignment mechanisms comprise a first radially-deployable alignment arm and a second radially-deployable alignment arm, and wherein aligning the probe comprises:
positioning the first radially-deployable alignment arm in the first end of the generally elongated heart valve opening; and
positioning the second radially-deployable alignment arm in the second end of the generally elongated heart valve opening.

18. The method of claim 15, wherein the stabilizing device comprises a first vacuum lumen and a first vacuum port, and stabilizing the leaflet tissue comprises applying a vacuum to the first vacuum port via the first vacuum lumen.

19. The method of claim 18, wherein the heart valve is a mitral valve having a first leaflet and a second leaflet, wherein the stabilizing device comprises a second vacuum lumen and a second vacuum port, wherein the second vacuum port is on an opposite side of the probe from the first vacuum port, and aligning the probe comprises:
positioning the first vacuum port adjacent the first leaflet; and
positioning the second vacuum port adjacent the second leaflet.

20. The method of claim 19, wherein stabilizing the leaflet tissue comprises:
stabilizing the first leaflet by applying vacuum to the first vacuum port; and
stabilizing the second leaflet by applying a vacuum to the second vacuum port.
